(19) United States
(12) Patent Application Publication
(10) Pub. No.: US 2022/0387533 A1
(43) Pub. Date: Dec. 8, 2022

(54) COMPOSITIONS AND METHODS FOR DIETARY MANAGEMENT OF IMMUNE HEALTH

(71) Applicant: Patients Medical, Inc., New York, NY (US)

(72) Inventor: Rashmi Gulati, New York, NY (US)

(73) Assignee: Patients Medical, Inc., New York, NY (US)

(21) Appl. No.: 17/825,386

(22) Filed: May 26, 2022

Related U.S. Application Data
(60) Provisional application No. 63/248,377, filed on Sep. 24, 2021.
(60) Provisional application No. 63/193,850, filed on May 27, 2021.

(51) Int. Cl. A61K 36/064; A61K 33/04; A61K 31/593; A61K 33/30; A61K 31/375; A61K 31/4045; A61K 31/198; A61K 31/352; A61K 31/122; A61K 38/06; A61K 31/197; A61K 35/745; A61K 31/519; A61K 33/06; A61K 31/714; A61K 9/00; A61K 33/00; A61K 31/685; A61K 31/205; A61P 31/14; A61K 35/747; C12Q 1/6869

(52) U.S. Cl. CPC A61K 36/064; A61K 33/04; A61K 31/593; A61K 33/30; A61K 31/375; A61K 31/4045; A61K 31/198; A61K 31/352; A61K 31/122; A61K 38/063; A61K 31/197; A61K 35/745; A61K 31/519; A61K 33/06; A61K 31/714; A61K 9/0019; A61K 33/00; A61K 31/685; A61K 31/205; A61P 31/14; A61K 35/747; C12Q 1/6869; C12Q 2600/156

(57) ABSTRACT

Aspects of the disclosure relate to compositions and methods for dietary management of certain conditions, such as infectious diseases. The disclosure is based, in part, on methods of identifying one or more single nucleotide polymorphisms (SNPs) in a subject and preparing a personalized report recommending a mixture of nutritional supplements and/or dietary aids based upon the presence of the SNPs. In some embodiments, the disclosure provides personalized compositions comprising nutritional supplements for dietary management of immune health and COVID support.

### RELATED APPLICATIONS

This application claims the benefit under 35 U.S.C. § 119(e) of the filing date of U.S. provisional Application Ser. No. 63/248,377, filed Sep. 24, 2021, and U.S. provisional Application Ser. No. 63/193,850, filed May 27, 2021, the entire contents of each of which are incorporated by reference herein.

### BACKGROUND

Corona viruses, such as SARS-CoV-2, and their resultant mutations and then double mutations, affect each person differently. Affected individuals in this epidemic have reported having many varied symptoms from a mild version to being hospitalized and in many cases thousands of resultant deaths worldwide. As the COVID-19 pandemic has progressed, it has become clear that many survivors even those who had mild cases continue to manage a variety of health problems long after the initial infection should have resolved. In what is believed to be the largest comprehensive study of long COVID-19 to date, researchers at Washington University School of Medicine in St. Louis in April 2021, showed that COVID-19 survivors including those not sick enough to be hospitalized have an increased risk of death in the six months following diagnosis with the virus. More than 30 million Americans have been infected with this virus and given that the burden of long COVID-19 is substantial, the lingering effects of this disease will reverberate for many years and even decades.

### SUMMARY

Aspects of the disclosure relate to compositions and methods for dietary management of certain conditions, such as infectious diseases (e.g., SARS-CoV-2 and variants thereof). The disclosure is based, in part, on methods of identifying one or more single nucleotide polymorphisms (SNPs) in a subject and preparing a personalized report recommending a mixture of nutritional supplements and/or dietary aids based upon the presence of the SNPs. In some embodiments, the disclosure provides personalized compositions comprising nutritional supplements for dietary management of immune health.

Accordingly, in some aspects, the disclosure provides a method for dietary management of immune health in a subject, the method comprising detecting in nucleic acid sequencing data obtained from the subject one or more single nucleotide polymorphisms (SNPs) in an alpha-1 antitrypsin (A1AT) gene; and providing a report indicating that the subject should ingest a selenium supplement as part of the subject's daily diet.

In some aspects, the disclosure provides a method for diagnosing a subject as having an increased risk of developing a pathogenic infection the method comprising detecting in nucleic acid sequencing data obtained from the subject one or more single nucleotide polymorphisms (SNPs) in: an alpha-1 antitrypsin (A1AT) gene; and two or more genes selected from ABCA7, ACE, AKT1, ANK3, APOE, BDNF, BNP, CACNA1C, CD33, CHRNA5/A3, CLOCK, COMT, CRHR1, CRP, FKBP5, FTP, FUT2, HDAC9, HLA-DQ2.2/3/2.5/8, HLA-DQB1, IL6, LRP1, LRRK2, MC4R, MEIS1, MIR-181, MTHFR, OXTR, PCSK9, PPARG, SMARCA4, and TREM2; and identifying the subject as having an increased risk of developing a pathogenic infection when the SNP in AAT is a SNP associated with AAT deficiency and when the SNP.

In some embodiments, a subject is a human.

In some embodiments, a SNP in the A1AT gene results in the subject expressing a PI*S or PI*Z A1AT protein. In some embodiments, one or more of the following SNPs are detected: ABCA7 (rs3764650), ACE (rs4343), AKT1 (rs2494732), ANK3 (rs10994336), APOE (rs429358, rs4712), BDNF (rs6265), BNP (rs198389), CACNA1C (rs1006737), CD33 (rs3865444), CHRNA5/3 (rs16969968, rs578776, rs588765), CLOCK (rs1801260), COMT (rs4680), CRHR1 (rs110402), CRP (rs1130864), FKBP5 (rs1360780), FTO (rs9939609), FUT2 (rs1047781, rs601338), HDAC9 (rs11984041), HLA-DQB1*06.02; HLA-DQ2.5 (rs2187668), HLA-DQ2.2 (rs2395182, rs7775228), HLADQ4 (rs4713586), HLA DQ8 (rs7454108), IL6 (rs1800795), LRRK2 (rs34637584), LRP1 (rs11172113), MC4R (rs17782313), MEIS1 (rs2300478), MIR-181 (rs322931), MTHFR (rs1801131, rs1801133), OXTR (rs53576), PCSK9 (rs11206510), PPARG (rs1801282), SMARCA4 (LDLR; rs1122608), and TREM2 (rs75932628).

In some embodiments, nucleic acid sequencing data comprises DNA expression data, RNA expression data, whole exome sequencing expression data, or microarray data. In some embodiments, nucleic acid sequencing data is obtained from a biological sample obtained from the subject. In some embodiments, a biological sample comprises blood, saliva, urine, feces, semen, or a tissue sample.

In some embodiments, SNPs are detected in 3, 4, 5, 6, 7, 8, 9, 10, 11, 12, 13, 14, 15, 16, 17, 18, 19, 20, 21, 22, 23, 24, 25, 26, 27, 28, 29, or 30 of the genes.

In some embodiments, the method further comprises producing a report indicating that the subject should be administered a nutritional supplement comprising selenium based upon the detecting of the A1AT SNP.

In some embodiments, selenium comprises selenized yeast (selenium yeast). In some embodiments, a nutritional supplement further comprises one or more of the following: vitamin D, N-acetylcysteine, zinc, melatonin, and vitamin C.

In some embodiments, the method further comprises providing to the subject a nutritional supplement comprising selenium. In some embodiments, the method further comprises administering a nutritional supplement comprising selenium to the subject.

In some aspects, the disclosure provides a nutritional composition comprising selenium, vitamin D3, zinc, vitamin C, melatonin, and N-acetylcysteine (NAC).

In some embodiments, the amount of selenium ranges from about 1 μg to about 500 μg.

In some embodiments, the selenium comprises selenized yeast (selenium yeast).

In some embodiments, the amount of vitamin D3 ranges from about 1 μg to about 100 μg or from about 1000 to 5000 International Units (IU).

In some embodiments, the amount of NAC ranges from about 1 mg to about 1200 mg.

In some embodiments, the amount of zinc ranges from about 1 mg to about 100 mg.

In some embodiments, the amount of melatonin ranges from about 1 mg to about 12 mg.

In some embodiments, the amount of vitamin C ranges from about 1 μg to about 1200 μg.

In some embodiments, the nutritional composition is a solid. In some embodiments, the nutritional composition is a powder. In some embodiments, the nutritional composition is in the form of a tablet. In some embodiments, the nutritional composition is contained within a capsule.

In some embodiments, the nutritional composition further comprises one or more pharmaceutically acceptable excipients.

In some embodiments, the nutritional composition comprises (or consists of) the formula listed in FIG. 1.

In some aspects, the disclosure provides a method of dietary management of a respiratory disease (e.g., SARS-CoV-2 or chronic obstructive pulmonary disease (COPD)), the method comprising administering a nutritional composition as described herein to a subject in need thereof.

In some embodiments, the subject has or is suspected of having an infection with SARS-CoV-2 or COPD. In some embodiments, the subject is known to have been exposed to SARS-CoV-2. In some embodiments, the subject is at risk of being exposed to SARS-CoV-2.

In some embodiments, the subject has one or more mutations in an A1AT gene. In some embodiments, the subject has A1AT deficiency.

In some aspects, the disclosure provides a nutritional composition comprising selenium, vitamin D3, zinc, vitamin C, quercetin, Coenzyme Q10, L-lysine, L-glutathione, and vitamin B5.

In some embodiments, the composition further comprises one or more probiotic bacteria.

In some embodiments, the composition is a solid. In some embodiments, the composition is a powder or a tablet.

In some embodiments, the formula comprises the formula listed in FIG. 2.

In some aspects, the disclosure provides a method for dietary management of a respiratory disease (e.g., SARS-CoV-2 or COPD), the method comprising administering a nutritional composition comprising selenium, vitamin D3, zinc, vitamin C, quercetin, Coenzyme Q10, L-lysine, L-glutathione, and vitamin B5 to a subject in need thereof.

Aspects of the disclosure are related to nutritional compositions (e.g., liquid nutritional compositions) that are useful for management of Long Haul COVID. In some aspects, the disclosure provides a liquid nutritional composition comprising water, ascorbic acid, B-complex, Calcium gluconate, Dexpanthenol, Folic acid, Magnesium chloride, Methylcobalamin, Multi-trace 4, Potassium chloride, Selenium, Sodium chloride (also solution, 0.9%), Zinc sulfate, NAC, and L-carnitine.

In some embodiments, the composition further comprises glutathione. In some embodiments, the composition further comprises phosphatidylcholine.

In some embodiments, the composition is formulated for intravenous (IV) delivery.

In some embodiments, the liquid composition comprises the formula listed in Table 2.

In some aspects, the disclosure provides a system for dietary management of a respiratory disease (e.g., SARS-CoV-2 or COPD) comprising: a first intravenous (IV) injection solution comprising B-complex, Calcium gluconate, Dexpanthenol, Folic acid, Magnesium chloride, Methylcobalamin, Multi-trace 4, Potassium chloride, Selenium, Sodium chloride (also solution, 0.9%), Zinc sulfate, NAC, and L-carnitine; a second intravenous (IV) injection solution comprising glutathione; and a third intravenous (IV) injection solution comprising phosphatidylcholine.

In some aspects, the disclosure provides a method for dietary management of Long Haul COVID, the method comprising administering a liquid nutritional composition or the solutions of system as described herein, to a subject in need thereof.

### BRIEF DESCRIPTION OF THE DRAWINGS

FIG. 1 shows one embodiment of a label for a nutritional composition as described by the disclosure.

FIG. 2 shows one embodiment of a label for a nutritional composition as described by the disclosure.

### DETAILED DESCRIPTION

The disclosure relates, in some aspects, to compositions and methods for dietary management of certain conditions (e.g., infectious diseases, respiratory illnesses, etc.). In some embodiments, the disclosure describes methods of identifying one or more single nucleotide polymorphisms (SNPs) in a subject and preparing a personalized report recommending a mixture of nutritional supplements and/or dietary aids based upon the presence of the SNPs. The disclosure is based, in part, on the recognition that subjects having certain SNPs, for example SNPs associated with A1AT deficiency, carry an increased risk of becoming infected with SARS-CoV-2, and that administration of compositions that comprise selenium facilitate dietary management of immune health that may mitigate such risk.

### Subjects

Aspects of the disclosure relate to identifying one or more SNPs in a biological sample obtained from a subject. As used herein, the term “subject” means any mammal, including mice, rabbits, and humans. In one embodiment, the subject is a human or non-human primate. The terms “individual” or “subject” may be used interchangeably with “patient.” In some embodiments, the biological sample may be any sample from a subject known or suspected of having a pathogenic infection (e.g., a bacterial infection, viral infection, etc.). In some embodiments, a biological sample is obtained from a subject having or at risk of becoming infected with a pathogenic agent, for example a coronavirus such as SARS-CoV-2 or a variant thereof.

The biological sample may be from any source in the subject's body including, but not limited to, any fluid (e.g., blood (e.g., whole blood, blood serum, or blood plasma), saliva, tears, synovial fluid, cerebrospinal fluid, pleural fluid, pericardial fluid, ascitic fluid, and/or urine), hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue). The biological sample may be any type of sample including, for example, a sample of a bodily fluid, one or more cells, a piece of tissue, or some or all an organ. In some embodiments, a biological sample comprises a blood sample. In some embodiments, a biological sample comprises a tissue sample. In some embodiments, the tissue sample is obtained by a buccal swab.

A subject “having” a particular disease or condition (e.g., a pathogenic infection, such as SARS-CoV-2 or a variant thereof) refers to a subject that exhibits one or more signs or symptoms of that disease or condition. In some embodiments, a subject having a pathogenic infection exhibits one or more of the following signs: fever, increased white blood cell count, aches, or respiratory symptoms (e.g., difficulty breathing, pulmonary fibrosis, pulmonary edema, etc.). In some embodiments, a subject having a pathogenic infection has been diagnosed as having the pathogenic infection (e.g., SARS-CoV-2 or a variant thereof) by a clinician (e.g., physician) and/or has received a positive result of a laboratory test that indicates the subject as having the pathogenic infection (e.g., SARS-CoV-2 or a variant thereof).

A subject “suspected of having” a particular disease or condition (e.g., a pathogenic infection, such as SARS-CoV-2 or a variant thereof) is a subject that exhibits one or more signs or symptoms of the disease or condition but has not been diagnosed by a clinician as having the disease or condition.

A subject “at risk of having” or “at risk of developing” a disease or condition may or may not exhibit one or more signs or symptoms of the disease or condition but may comprise one or more genetic mutations (e.g., SNPs) that increases the risk that the subject will develop the disease or condition (e.g., relative to a normal healthy subject not having such mutations). In some embodiments, a subject having one or more mutations in the A1AT gene (also referred to as AAT) has an increased risk of being infected with certain pathogens, for example respiratory pathogens including SARS-CoV-2 or variants thereof. In some embodiments, a subject having an “increased risk of developing” a particular disease or condition (e.g., a pathogenic infection, such as SARS-CoV-2 or a variant thereof) has an increased risk of developing the disease or infection relative to a normal, healthy subject. In some embodiments, a subject having an “increased risk of developing” a particular disease or condition (e.g., a pathogenic infection, such as SARS-CoV-2 or a variant thereof) has an increased risk of developing the disease or infection relative to a subject characterized as not having one or more SNPs (e.g., one or more SNPs in an AAT gene) in a gene that the subject does have one or more SNPs.

The level of increased risk may vary. In some embodiments, a subject having an increased risk of developing a disease or infection may be at least 5%, 10%, 20%, 30%, 40%, 50%, 60%, 70%, 80%, 90%, or 100% more likely to develop the disease or infection relative to a normal, healthy subject (or a subject lacking the one or more SNPs of the subject). In some embodiments, a subject having an increased risk of developing a disease or infection is more than 100% likely (e.g., at least 150%, 200%, 300%, 500%, 1000%, or more) to develop the disease or infection relative to a normal, healthy subject (or a subject lacking the one or more SNPs of the subject).

### Single Nucleotide Polymorphisms (SNPs)

Aspects of the disclosure relate to methods for identifying single nucleotide polymorphisms (SNPs) in genes of a subject by analyzing gene expression data obtained from a biological sample that has been obtained from the subject. A “single nucleotide polymorphism” or “SNP” refers to a single nucleotide change that causes a nucleic acid sequence for a gene to differ between members of a species or paired chromosomes in an individual. A SNP may result in an insertion, deletion, or substitution of the nucleotide. In some embodiments, a substitution is non-synonymous (e.g., the substitution does result in a change in the corresponding amino acid sequence encoded by the nucleic acid sequence). In some embodiments, a non-synonymous substitution results in a missense or non-sense change in the corresponding amino acid sequence.

The presence of one or more SNPs in a subject may be measured by any suitable method. Examples of methods for detecting SNPs in a biological sample include but are not limited to hybridization-based methods (e.g., dynamic allele-specific hybridization (DASH) genotyping, use of molecular beacons, SNP microarrays, etc.), enzymatic methods (e.g., restriction fragment length polymorphism, PCR-based reactions, Flap endonuclease-based reactions, primer extension assays, Taq-man assays, etc.), and post-amplification detection methods, such as temperature gradient gel electrophoresis, denaturing high performance liquid chromatography (DHPLC), next-generation sequencing, nanopore sequencing, etc.

In some embodiments, detecting the presence of one or more SNPs in a biological sample comprises obtaining sequencing data (e.g., sequencing data previously obtained from a biological sample taken from the subject) and subjecting the sequencing data to one or more bioinformatics methods or analysis pipelines to detect the SNPs. In some embodiments, the one or more bioinformatics methods comprises inputting the SNPs into an artificial intelligence (AI)-based probabilistic model in order to identify suitable nutritional supplements.

The disclosure is based, in part, on the recognition that detection of SNPs in a group of genes of a subject may be indicative of that subject having a higher risk of being infected (or having a severe infection) with certain pathogenic agents, for example SARS-CoV-2 or variants thereof. In some embodiments, a SARS-CoV-2 virus comprises the sequence set forth in NCBI Accession Number NC_045512. In some embodiments a subject is infected with a variant of a SARS-CoV-2 virus. Examples of variants include but are not limited to B.1.1.7, B.1.351, P.1, B.1.427, B.1.429, and B1.617.

In some embodiments, methods described herein comprise detecting SNPs in one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or more) of the following genes of a subject: AAT, ABC7A, ACE, AKT1, ANK3, APOE, BDNF, BNP, CACNA1C, CD33, CHRNA5/A3, CLOCK, COMT, CRHR1, CRP, FKBP5, FTP, FUT2, HDAC9, HLA-DQ2.2/3/2.5/8, HLA-DQB1, IL6, LRP1, LRRK2, MC4R, MEIS1, MIR-181, MTHFR, OXTR, PCSK9, PPARG, SMARCA4, and TREM2. Each of the listed genes is described in further detail below.

Aspects of the disclosure relate to methods for identifying one or more SNPs in an AAT gene of a subject. Alpha-1 antitrypsin (AAT), also known as serpin peptidase inhibitor, clade A (SERPINA1), is a protein that functions as proteinase (protease) inhibitor. AAT is one of the primary circulating serum anti-proteases in humans. Several SNPs present in the AAT gene have been observed to be associated with disease in humans. For example, North American and Northern European populations have been observed to possess at least one copy of a mutant allele, known as PI*Z (Z-AAT) which results from a single amino acid substitution of lysine for glutamate at position 342 in the mature protein (position 366 in the precursor protein). In some embodiments, a subject possesses a different mutant allele, PI*S (S-AAT), which results from a single substitution of valine for glutamic acid at position 264. In the homozygous state, such SNPs may lead to severe deficiency of AAT in the subject. As used herein the term, “alpha-1 antitrypsin deficiency” refers to a condition resulting from a deficiency of functional AAT in a subject. Insufficient production of AAT or expression of mutant AAT may result in damage to a subject's lung and/or liver, and in some embodiments places the subject at an increased risk of developing certain pathogenic infections, such as SARS-CoV-2.

In some embodiments, a SNP in the A1AT gene results in the subject expressing a PI*S or PI*Z A1AT protein. In some embodiments, a subject expressing PI*S or PI*Z A1AT protein is characterized as having A1AT deficiency. In some embodiments, subjects having A1AT deficiency have an increased risk of developing an infection (e.g., a severe infection) with the SARS-CoV-2 virus. Examples of AAT SNPs include but are not limited to one or more SNPs located in the SERPINA gene cluster on 14q32.13, rs17580, and rs28929474.

In some embodiments, one or more SNPs are detected in an ABCA7 gene. ABCA7 encodes ATP-binding cassette A7 protein. In the brain, ABCA7 is highly expressed in hippocampal CAl neurons and microglial cells. ABCA7 is thought to play a role in Alzheimer's disease (AD) through three primary means. First, ABCA7 contributes to efflux of phospholipids and cholesterol, which is hypothesized to be related to AD pathogenesis through amyloid B related pathways. Second, ABCA7 plays a significant role in regulating phagocytosis of apoptotic cell debris. It is highly expressed in microglial cells in which ABCA7 knockdown leads to ineffective engulfment of apoptotic debris. Finally, ABCA7 may be directly involved in amyloid B homeostasis. In some embodiments, the G allele of the ABCA7 variant is associated with an approximate 17-20% increased likelihood of developing Alzheimer's disease (AD) with one risk allele (C/G)3833. In some embodiments, methods described herein comprise detecting one or more G alleles of ABCA7. In some embodiments, the SNP in ABCA7 is rs3764650.

In some embodiments, one or more SNPs are detected in an ACE gene. The angiotensin I-converting enzyme (ACE), encoded by the ACE gene, plays a role in the production of angiotensin II vasoconstrictor and the catabolism of bradykinin and kallidin (e.g., vasodilators). The ACE enzyme is a key component of the renin-angiotensin system, a major pathway and therapeutic target involved in the pathogenesis of hypertension and cardiovascular disease. In some embodiments, the one or more SNPs detected in ACE comprises a G2350A SNP. This SNP has been observed to be associated with an 18-25% increased odds of developing cardiovascular disease, including cerebrovascular events and coronary heart disease. In some embodiments, the ACE A allele confers 39% increased odds of metabolic syndrome, which consists of a cluster of symptoms including obesity, hyperlipidemia, and insulin resistance. In some embodiments, the SNP in ACE is rs4343.

In some embodiments, one or more SNPs are detected in a PKB gene (also referred to as AKT1 gene). This gene encodes one of three isoforms of protein kinase B (PKB). PKB participates in the signaling of multiple G-protein coupled receptors, including dopamine signaling, such that decreased AKT function results in increased dopamine receptor stimulation.

In some embodiments, PKB variants have been associated with development of psychotic symptoms in habitual marijuana users. In some embodiments, the one or more SNPs detected in PKB comprises rs2498794. In some embodiments, the SNP comprises a C allele. In some embodiments, the C/C genotype has been associated with a two-fold increase in risk of psychotic symptom development in cannabis users. In some embodiments, the SNP in AKT1 is rs2494732.

In some embodiments, one or more SNPs are detected in an ankyrin 3 (ANK3) gene. ANK3 belongs to a family of scaffolding proteins known as the ankyrins and plays a role in the maintenance of sodium ion channels. In some embodiments, the SNP detected in an ANK3 gene comprises rs994336. In some embodiments, the SNP comprises a T allele. GWAS analyses have observed a correlation between this variation and disorders characterized by mood instability and lability, with an effect size of 18% in a meta-analysis for mood lability. Many studies indicate that this variant is associated with changes in anatomical connections that may be related to cognitive and affective symptoms. More specifically, this variation has been associated with anhedonia, altered novelty seeking, impaired threat/stress signal processing, poorer cognition, an impact on working memory, and reduced integrity of white matter tracts. In some embodiments, the SNP in ANK3 is rs10994336.

In some embodiments, one or more SNPs are detected in an Apolipoprotein E (APOE) gene. Apolipoprotein (ApoE) is a multifunctional protein with central roles in lipid metabolism, neurobiology, and neurodegenerative diseases. ApoE has three major isoforms (E2, E3, and E4) with different effects on lipid, neuronal, and vascular homeostasis. The E2 allele is the rarest form of APOE. In some embodiments, a subject having one copy of the E2 allele appears to reduce the risk of developing Alzheimer's by up to 40%. The E3 allele is the most common. The E4 allele, present in approximately 10-15% of people, has been observed to be associated with an increased risk for Alzheimer's disease and lowers the age of disease onset. In some embodiments, having one copy of the E4 allele (E4/E3 or E4/E2) can increase risk by 2 to 3 times while two copies (E4/E4) can increase the risk by approximately 12 to 15-fold. In some embodiments, the E4 variant is also associated with hypertension and cerebral infarction. In some embodiments, one or more SNPs in APOE are located at Ch.19q3. In some embodiments, the SNP in APOE is rs429358 or rs4712.

In some embodiments, one or more SNPs are detected in a Brain-derived neurotrophic factor (BDNF) gene. BDNF regulates neuronal differentiation, migration, survival, and synaptic plasticity. In some embodiments, BDNF gene is expressed in the prefrontal cortex (PFC) and hippocampus of a subject. In some embodiments, BDNF has been implicated to play a role in the etiology of several psychiatric disorders and the biology of learning and memory. In some embodiments, the one or more SNPs detected in BDNF comprise a Val66Met polymorphism, which has been observed to be associated with reduced BDNF secretion, depression, and altered stress reactivity. In some embodiments, a recommendation of physical activity may improve cognition and working memory to a greater degree in Met allele carriers and may be implemented as therapy, if clinically indicated. In some embodiments, the SNP in BDNF is rs6265.

In some embodiments, one or more SNPs are detected in a brain natriuretic peptide (BNP) gene, also referred to as NPPB. BNP plays a role in regulation of circulatory volume, plasma renin-aldosterone concentrations, natriuresis, and maintenance of blood pressure. Elevated serum BNP levels stimulate natriuresis and diuresis and promote peripheral vasodilation. Plasma BNP and NT-proBNP levels have been used as biomarkers to aid in the diagnosis and prognosis of heart failure. In some embodiments, one or more of the SNPs detected in NPPB comprise a T381C polymorphism. In some embodiments, the T381C polymorphism is associated with significantly increased expression of BNP. Individuals with this polymorphism may benefit from this increased expression, as it is associated with approximately 20% decreased odds of developing hypertension, 15-15% decreased odds of type 2 diabetes, and decreased odds of developing cardiovascular disease. In some embodiments, the C/C genotype has been observed to be associated with lower rates of all-cause mortality and cardiovascular death in subjects. In some embodiments, the SNP in BNP is rs198389.

In some embodiments, one or more SNPs are detected in a Cay1.2 Voltage-Dependent L-Type Calcium Channel, a1C subunit (CACNA1C) gene. CACNA1C is important in the regulation of calcium signaling; it encodes for an alpha-1 subunit of a voltage dependent calcium ion channel. In some embodiments, one or more SNPs detected in a CACNA1C gene comprise an A allele. In some embodiments, the A allele has been observed to be associated with conditions related to mood instability and lability, with an effect size up to 20%. In some embodiments, one or more SNPs detected in a CACNA1C gene comprise rs1006737. It has also been reported that this variant is associated with changes in amygdala volume, frontal-hippocampal function, and has been hypothesized to be related to glutamate signaling.

In some embodiments, one or more SNPs are detected in a Cluster of Differentiation 33 (CD33) gene. CD33 (also referred to as Siglec-3) encodes a transmembrane glycoprotein that is expressed on the surface of microglia and is an essential regulator of cell activity. Microglial cells are found in one of two states: nonphagocytic or neuroinflammatory. The nonphagocytic state increases microglial clearance of amyloid and other debris, whereas the neuroinflammatory state leads to oxidative stress and neuronal damage. CD33 is typically thought of as an “off switch” for the nonphagocytic state. In some embodiments, one or more SNPs detected in a CD33 gene comprise an A allele. In some embodiments, the A allele (resilient allele) of the CD33 variant has been observed to confer reduced surface expression of CD33. Thus, the ratio of nonphagocytic to neuroinflammatory microglial cells will be higher, with increased clearance of amyloid and decreased risk of Alzheimer's disease. In some embodiments, the C>A substitution confers an approximate 10% reduced risk of developing late onset Alzheimer's disease. In some embodiments, the SNP in CD33 is rs12459419 or rs3865444.

In some embodiments, one or more SNPs are detected in a Cholinergic Receptor Nicotinic Alpha 5, Alpha 3 Subunit gene (CHRNAS3 or CHRNAS5). Nicotine selectively binds to nicotinic acetylcholine receptors (nAChRs), partially encoded by CHRNA5 and CHRNA3, which open ion channels to exert physiological effects. The receptors activate neurons in the dopaminergic reward pathway and can facilitate nicotine dependence and addiction with chronic exposure. In some embodiments, the one or more SNPs detected in a CHRNAS3 or CHRNAS5 gene comprises rs16969968 (D398N). In some embodiments, the rs16969968 (D398N) polymorphism is associated with reduced receptor function. In some embodiments, the A allele at rs16969968 has been observed to be associated with an increased risk for nicotine dependence. In some embodiments, the one or more SNPs detected in a CHRNAS3 or CHRNAS5 gene comprises rs578776 or rs588765. In some embodiments, rs578776 and rs588765 have been observed to affect the mRNA expression levels of CHRNA5. In some embodiments, individuals that are homozygous for CHRNA3 or CHRNS5 risk alleles may have a higher chance of relapsing following smoking cessation. In some embodiments, the minor allele (A) at rs578776, is associated with protection against nicotine dependence.

In some embodiments, one or more SNPs are detected in a Circadian Locomotor Output Cycles Kaput (CLOCK) gene. CLOCK is one of the core genes involved in the “molecular clock,” governing circadian rhythm in the human body. CLOCK encodes the CLOCK transcription factor, which is involved in a transcriptional/translational feedback loop encompassing multiple genes that work to regulate circadian activity across several biological functions, including energy balance and metabolism. In some embodiments, CLOCK variants are associated with obesity and eating behavior traits. In some embodiments, the one or more SNPs detected in a CLOCK gene comprise a T3111C variant. In some embodiments, the T3111C variant confers increased expression, as well as an increased risk of obesity and metabolic syndrome in a subject. In some embodiments, the T3111C allele is associated with diurnal preference and emotional eating behavior, both of which are postulated to play a role in an observed increased risk of metabolic syndrome. In some embodiments, the SNP in CLOCK is rs1801260.

In some embodiments, one or more SNPs are detected in a Catechol-O-Methyltransferase (COMT) gene. COMT is an enzyme responsible for breakdown of dopamine, particularly in the frontal lobes of the brain, a critical area for memory, attention, judgement, and other executive functions. In some embodiments, one or more SNPs detected in a COMT gene comprise a Val66Met variant. In some embodiments, the Val66Met variant affects functional capacity of the enzyme to degrade dopamine. In some embodiments, the Met allele results in reduced enzymatic activity, while the Val allele results in increased activity. In some embodiments, patients who have normal levels of dopamine degradation possess one increased and one decreased function allele (Val/Met). In some embodiments, patients with the Val/Val genotype display elevated enzyme activity and increased dopamine degradation. In some embodiments, patients with the Met/Met genotype display reduced enzyme activity and decreased dopamine degradation. Clinical studies have shown that the Val/Val genotype may have negative influence regarding cognitive function, memory retention, motivation, and judgement. Studies have shown that individuals with the Met/Met genotype may perform better on cognitive tasks, including working memory and reward-learning but may also be at increased risk of cognitive perseveration, PTSD, OCD (in men), and addictive behavior. In some embodiments, the SNP in COMT is rs4680.

In some embodiments, one or more SNPs are detected in a Corticotropin-Releasing Hormone Receptor (CRHR1) gene. CRHR1 encodes for the corticotropin-releasing hormone (CRH) receptor, which contributes to the hypothalamic-pituitary axis (HPA) modulation of neuroendocrine and behavioral responses to stress. CRHR1 is essential for the activation of signal transduction pathways that activate mesolimbic and HPA responses to many types of stress. In some embodiments, one or more SNPs detected in a CRHR1 gene comprises a G allele at Ch17q21-22. In some embodiments, the G allele is associated with significantly increased cortisol in response to stress, poor working memory, and increased rates of adult depression in the context of childhood trauma or maltreatment. In some embodiments, G/G homozygous individuals have up to a two-fold increase in depressive symptoms versus A/A homozygous individuals with a history of moderate-severe child abuse. In some embodiments, the SNP in CRHR1 is rs1876828, rs242939, rs242941, or rs110402.

In some embodiments, one or more SNPs are detected in a C reactive protein (CRP) gene. CRP encodes for C-reactive protein, an acute phase reactant that is produced in response to inflammation and tissue damage and is a predictor of cardiovascular disease. In some embodiments, one or more SNPs detected in a CRP gene comprise the CT genotype or TT genotype of rs1130864. In some embodiments, subjects having these genotypes have 1.36 or 2.31 (respectively) greater odds of significantly elevated serum CRP (23 mg/1) than CC allele carriers.

In some embodiments, one or more SNPs are detected in a FK506-Binding Protein 5 (FKBP5) gene. Stress response is regulated by the hypothalamic-pituitary-adrenal (HPA) axis. Activation of the HPA triggers release of cortisol from the adrenal cortex, which enters the bloodstream to act on target organs thereby facilitating the “Fight or Flight” response. This process is tightly regulated by a negative feedback loop involving glucocorticoid receptors (GR). FKBP5 is a negative regulator of GR function, and changes in FKBP5 expression can have significant effects on HPA function. In some embodiments, one or more SNPs detected in a FKBP5 gene comprise a T allele. In some embodiments, the T allele confers increased expression of the protein and dysregulated cortisol response to stress. This has been shown to potentiate the risk of depression and posttraumatic stress disorder following traumatic experiences such as injury or childhood maltreatment. In some embodiments, TT individuals have up to an eight-fold increased risk of major depressive disorder (MOD) when exposed to physical abuse, relative to CC individuals. In some embodiments, the SNP in FKBP5 is rs1360780, rs3800313, or rs9296158.

In some embodiments, one or more SNPs are detected in an Alpha-Ketoglutarate-Dependent Dioxygenase (FTO) gene. The FTO gene encodes an alpha-ketoglutarate-dependent dioxygenase, which is responsible for methylation of amino acid and RNA nucleosides. In some embodiments, one or more SNPs detected in a FTO gene comprise a T>A variant. In some embodiments, the T>A variant is associated with metabolic syndrome for example a change in BMI per copy of the A allele relative to patients having a T allele. In some embodiments, the A allele is associated with increased risk of metabolic syndrome, including increased risk of obesity, type 2 diabetes, and cardiovascular disease. In some embodiments, the SNP in FTO is rs9939609.

In some embodiments, one or more SNPs are detected in a fucosyltransferase 2 (FUT2) gene. FUT2 has been observed to affect the composition of intestinal microbiota due to the addition of an important fucose moiety to luminal surface receptors, which allow bacterial attachment and growth. In some embodiments, one or more SNPs detected in a FUT2 gene comprise a rs1047781 A>T SNP or a rs601338 G>A SNP. In some embodiments, the detected SNP in FUT2 is associated with “non-secretor” status, meaning that the variant confers altered fucosyltransferase activity in the subject. The non-secretor genotype typically confers altered vitamin B12 absorption and a 7-14% increased risk of inflammatory GI conditions including Crohn's disease, primary sclerosing cholangitis, celiac disease, or inflammatory bowel disease. This is likely due to dysregulation of gut microbiota.

In some embodiments, one or more SNPs are detected in a Histone Deacetylase 9 (HDAC9) gene. HDAC9 is a member of a large family of genes that encode proteins responsible for deacetylation of histones and is therefore involved in the epigenetic modification and transcriptional regulation of DNA. HDAC9 typically inhibits myogenesis and is involved in cardiomyocyte and vascular smooth muscle cell development. In some embodiments, one or more SNPs detected in a HDAC9 gene comprise a C>T variant. In some embodiments, the C>T variant results in thickening of the carotid intima-media, which is an independent predictor of stroke. In some embodiments, the T allele is associated with a 24% increased likelihood of stroke relative to patients having other alleles. In some embodiments, the SNP in HDAC9 is rs2240419, rs2389995, or rs11984041.

In some embodiments, one or more SNPs are detected in a Major Histocompatibility Complex, Class II gene, for example a HLA-DQ2.2 gene, a HLA-DQ4 gene, a HLA-DQ2.5 gene, or a HLA-DQ8 gene. The human leukocyte antigen (HLA) haplotypes DQ2 and DQ8 (HLA-DQ2 and HLA-DQ8, respectively) are strongly associated with the development of celiac disease, although not all individuals positive for DQ2 and DQ8 develop the disease. These haplotypes refer to a set of HLA variants, which encode heterodimers of major histocompatibility complex (MHC) class II T cell antigens. Variants tend to lie in the antigen-binding site of these proteins, and the DQ2 and DQ8 haplotypes are key to binding gluten and mediating the immune response, allowing the development of celiac disease. In some embodiments, one or more SNPs detected in a HLA-DQ gene comprise a SNP associated with DQ2.5 haplotype, DQ2.2 haplotype, or a DQ8 haplotype. In some embodiments, each of these haplotypes confers an increased risk of celiac disease in a subject. The validity of these tagging SNPs is supported across numerous studies. In some embodiments, the SNP in a MHC class II T cell antigen is rs2187668, rs2395182 or rs7775228.

In some embodiments, one or more SNPs are detected in a Major Histocompatibility Complex, Class II gene, for example a HLA-DQB1 gene. Narcolepsy is a sleep disorder marked by excessive daytime sleepiness and cataplexy (muscle weakness or paralysis triggered by strong emotion. There is evidence that the disorder has an autoimmune etiology, as much of the strong genetic associations have been found in a region of the genome that includes genes associated with the immune system, including those related to the human leukocyte antigen (HLA) system. In some embodiments, one or more SNPs detected in a HLA-DQB1 gene. In some embodiments, individuals with narcolepsy carry the HLA-DQB10602 allele. In some embodiments, the HLA-DQB10602 allele has been shown to be associated with increased susceptibility for developing multiple sclerosis, another disease of the central nervous system, likely by a similar pathogenesis. In some embodiments, the SNP is rs2858884. In some embodiments, the SNP in HLADQ4 is rs4713586. In some embodiments, the SNP in HLA DQ8 is rs7454108.

In some embodiments, one or more SNPs are detected in an interleukin-6 (IL6) gene. Interleukin-6 is secreted by T cells and macrophages to stimulate the acute phase immune response and is a primary determinant of hepatic production of C-reactive protein, a marker of inflammation and tissue damage. The role of IL6 in the body is complex and has been classified as both a pro- and anti-inflammatory cytokine, depending on cellular context. In muscular tissue, it acts to acutely reduce inflammation, but may also play a role in the deleterious, proinflammatory cascade seen in chronic inflammation. In some embodiments, the one or more SNPs detected in an IL6 gene comprises a G174C polymorphism. In some embodiments, the G174C polymorphism confers reduced expression of IL-6. In some embodiments, healthy subjects having the CC genotype show significantly lower IL-6 plasma levels than G allele carriers. This variant has been implicated in a wide range of diseases with an inflammatory etiology, including vascular dementia, chronic arthritis, inflammatory bowel conditions, and fatigue. In some embodiments, C allele (resilient allele} carriers have reduced odds of developing periodontitis relative to G allele carriers. In some embodiments, the SNP in IL6 is rs1800795.

In some embodiments, one or more SNPs are detected in a Leucine Rich Repeat Kinase 2 (LRRK2) gene. The biological function of LRRK2 may be involved in regulating peroxidase activity, which is essential for preventing oxidative stress and neuronal degeneration. It is highly expressed in the putamen and substantia nigra, areas of the brain that are susceptible to degeneration in Parkinson's disease. In some embodiments, one or more SNPs detected in a LRRK2 gene comprise a G2019S variant. In some embodiments, the G2019S variant causes an amino acid change from glycine to serine, which may result in decreased peroxidase activity, oxidative cellular damage, and neurodegeneration. In some embodiments, the SNP in LRRK2 is rs10784486 or rs34637584. In some embodiments, the SNP in LRP1 is rs11172113.

In some embodiments, one or more SNPs are detected in a Melanocortin 4 Receptor (MC4R) gene. MC4R moderates the effects of leptin, a neuro-hormone involved with satiety, metabolism, and energy balance. Variants of MC4R influence body mass index (BMI) and the risk of obesity. In some embodiments, one or more SNPs detected in a MC4R gene comprise a T>C SNP. In some embodiments, the T>C variant is associated with increased BMI, with an increase in odds of the subject being obese. In some embodiments, C allele carriers may be particularly likely to reap the benefits of adhering to a Mediterranean diet to reduce the increased risk of diabetes conferred by this genotype. In some embodiments, the SNP in MC4R is rs17700633 or rs17782313.

In some embodiments, one or more SNPs are detected in a Meis Homeobox 1 (MEIS1) gene. MEIS1 encodes a homeobox protein that is part of a regulatory network, which is involved in spinal motor neuronal connectivity. In some embodiments, one or more SNPs detected in a MEIS1 gene comprise a T>G SNP. In some embodiments, subjects comprising the G allele have an increased risk of developing restless leg syndrome (RLS), a sensorimotor disorder characterized by unpleasant or uncomfortable sensations in the legs and an irresistible urge to move them, particularly at night. In some embodiments, the G allele increases likelihood of developing RLS. In some embodiments, the SNP in MEIS1 is rs2300478.

In some embodiments, one or more SNPs are detected in a microRNA-181 precursor (MIR181) gene. MicroRNAs (miR) modulate gene expression by regulating messenger RNA. miR-181 has been shown to be involved in the development of neurons by promoting spine formation and reducing dendrite branching and axon growth, possibly through an immunomodulatory mechanism. MiR-181 is expressed in the nucleus accumbens (NAc) and hippocampus, the brain regions involved with pleasure, reward, and motivation. In some embodiments, one or more SNPs detected in a MIR181 gene comprise an A allele. In some embodiments, the A allele of miR-181 is a resilience allele, conferring decreased expression of miR-181a/b in the brain. In some embodiments, the A allele is associated with greater NAc reactivity to positive emotional stimuli and enhanced fear inhibition and positive emotion reactivity and spiritual well-being. In some embodiments, the SNP in MIR-181 is rs322931.

In some embodiments, one or more SNPs are detected in a Methylenetetrahydrofolate Reductase (MTHFR) gene. MTHFR is an enzyme responsible for catalyzing the conversion of folic acid to methyl folate. Methyl folate is the active form of folic acid, a vital cofactor for the synthesis of norepinephrine, dopamine, and serotonin. In some embodiments, the one or more SNPs detected in a MTHFR gene comprise a T allele of the C677T variant or a C allele of the A1298C variant. In some embodiments, the C677T variant and the A1298C variant lead to reduced enzymatic activity of MTHFR, resulting in inefficient folic acid metabolism and production of methyl folate. In some embodiments, these variants are associated with increased risk of developing disorders characterized by depressed mood and mood lability. In some embodiments, the SNP in MTHFR is rs1801131 or rs1801133.

In some embodiments, one or more SNPs are detected in an oxytocin receptor (OXTR) gene. Oxytocin is a neuro-hormone involved in sociality and empathy in adults, as well as emotional bonding between parents and infants. In some embodiments, one or more SNPs detected in an OXTR gene comprise a G allele. In some embodiments, G allele homozygotes have greater measures of sociality than A allele carriers. In some embodiments, the SNP in OXTR is rs53576.

In some embodiments, one or more SNPs are detected in a Proprotein Convertase, Subtilisin/Kexin-Type 9 (PCSK9) gene. PCSK9 expresses an enzyme that is strongly linked with lipoprotein homeostasis. Downregulation of PCSK9 results in increased expression of low-density lipoprotein receptors, which subsequently results in increased metabolism (clearance) of cholesterol and LDL particles in the blood. In some embodiments, the one or more SNPs detected in a PSCK9 gene comprise a C allele of the T>C missense variant. In some embodiments, presence of the C allele results in decreased serum LDL and/or decreased likelihood of the subject developing coronary artery disease (CAD) and myocardial infarction (MI) R46L variant retroactive for MI. In some embodiments, the SNP in PCSK9 is rs11206510.

In some embodiments, one or more SNPs are detected in a Peroxisome Proliferator-Activated Receptor Gamma (PPARG) gene. PPARG encodes a nuclear receptor that regulates gene expression and affects peroxisome activity, playing a significant role in fatty acid oxidation and metabolism. In some embodiments, one or more SNPs detected in a PPARG gene comprise a G allele at rs1801282 (Pro12Ala). In some embodiments, the G allele has been observed to alter the ability of PPARG to bind target gene promoters, which effects multiple pathways involved in metabolism. In some embodiments, C/G and G/G genotypes are associated with decreased risk of type 2 and gestational diabetes compared to the C/C genotype. In some embodiments, the C/G and G/G genotypes confer increased odds of obesity. In some embodiments, individuals with C/G and G/G genotypes have a health advantage compared to those with C/C genotype when adhering to a Mediterranean diet. In some embodiments, G allele carriers show reduced telomere shortening and overall greater telomere length following dietary intervention with the Mediterranean diet. In some embodiments, individuals with the C/C genotype are more likely to benefit from physical training for weight loss.

In some embodiments, one or more SNPs are detected in a Catalytic Subunit of the SWI/SNF Chromatin-Remodeling Complex (SMARCA4) gene. The protein expressed by SMARCA4 is involved with epigenetic regulation, which can affect the rate of gene transcription. SMARCA4 lies near LDLR, the gene that expresses the low-density lipoprotein receptor. In some embodiments, one or more SNPs detected in SMARCA4 comprise a G>T polymorphism. In some embodiments, the G (major) allele confers increased risk of early-onset myocardial infarction and coronary artery disease in Caucasian and Asian subjects but not in those of African descent. In some embodiments, the SNP in SMARCA4 is rs11879293 or rs12232780 (and are protective for myocardial infarction). In some embodiments, the SNP in SMARCA4 (LDLR) is rs1122608.

In some embodiments, one or more SNPs are detected in a Triggering Receptor Expressed on Monocytes 2 (TREM2) gene. TREM2 is a microglial surface receptor that triggers intracellular protein tyrosine phosphorylation and plays an essential role in removing neural debris, Homozygous loss-of-function mutations in TREM2 have been associated with an autosomal recessive form of early-onset dementia. In some embodiments, one or more SNPs detected in a TREM2 gene confers an amino acid change from arginine to histidine at position 47 of the protein (R47H). In some embodiments, this SNP confers an increased risk of developing Alzheimer's disease in Caucasian subjects. In some embodiments, the SNP in TREM2 is rs75932628.

### Methods

Aspects of the disclosure relate to detecting one or more SNPs in a subject and then using artificial intelligence (AI) based probabilistic modeling to produce a report comprising one or more recommendations for dietary management of immune health of the subject.

As used herein, “dietary management” refers to the treatment of a disorder by administration of a medical food, food ingredient, or dietary supplement. As used herein, the terms “treatment,” “treating,” and “therapy” refer to therapeutic treatment and prophylactic or preventative manipulations. The terms further include ameliorating existing symptoms, preventing additional symptoms, ameliorating, or preventing the underlying causes of symptoms, preventing, or reversing causes of symptoms, for example, symptoms associated with a disease caused by the presence of one or more SNPs in a gene of a subject Thus, the terms denote that a beneficial result has been conferred on a subject with a disorder, or with the potential to develop such a disorder. Furthermore, the term “treatment” is defined as the application or administration of an agent (e.g., a nutritional composition) to a subject, who may have a disease, a symptom of disease or a predisposition toward a disease, with the purpose to cure, heal, alleviate, relieve, alter, remedy, ameliorate, improve, or affect the disease, the symptoms of disease or the predisposition toward disease.

In some embodiments, producing the report comprises the steps of (i) detecting (or identifying) the one or more SNPs in the subject (or a biological sample of the subject); (ii) inputting the SNPs into a computer model that correlates the presence of the SNPs with certain nutritional or dietary recommendations; and (iii) producing a report indicating a personalized nutritional composition for dietary management of conditions to which the subject is at increased risk of developing based upon the presence of the SNPs. In some embodiments, the one or more SNPs are selected from ABCA7 (rs3764650), ACE (rs4343), AKT1 (rs2494732), ANK3 (rs10994336), APOE (rs429358, rs4712), BDNF (rs6265), BNP (rs198389), CACNA1C (rs1006737), CD33 (rs3865444), CHRNA5/3 (rs16969968, rs578776, rs588765), CLOCK (rs1801260), COMT (rs4680), CRHR1 (rs110402), CRP (rs1130864), FKBP5 (rs1360780), FTO (rs9939609), FUT2 (rs1047781, rs601338), HDAC9 (rs11984041), HLA-DQB1*06.02; HLA-DQ2.5 (rs2187668), HLA-DQ2.2 (rs2395182, rs7775228), HLADQ4 (rs4713586), HLA DQ8 (rs7454108), IL6 (rs1800795), LRRK2 (rs34637584), LRP1 (rs11172113), MC4R (rs17782313), MEIS1 (rs2300478), MIR-181 (rs322931), MTHFR (rs1801131, rs1801133), OXTR (rs53576), PCSK9 (rs11206510), PPARG (rs1801282), SMARCA4 (LDLR; rs1122608), and TREM2 (rs75932628). In some embodiments, SNPs are referred to by their NIH RefSNP number, which refers to a locus accession for a variant type assigned by dbSNP. The RefSNP catalog is a non-redundant collection of submitted variants which were clustered, integrated and annotated. RefSNP number is the stable accession regardless of the differences in genomic assemblies. RefSNP numbers facilitate large-scale studies in association genetics, medical genetics, functional and pharmaco-genomics, population genetics and evolutionary biology, personal genomics, and precision medicine. They provide a stable variant notation for mutation and polymorphism analysis, annotation, reporting, data mining, and data integration.

In some embodiments the computer model comprises an AI-based probabilistic model application that comprehensively links at least three (e.g., 3, 4, 5, or more) National Institutes of Health (NIH) databases in order to match: 1) a patient's genetic data to predisposition to specific medical conditions, 2) matching those conditions to behaviors and nutritional supplements that will ameliorate those conditions, and 3) matching those supplements against all known contraindicated supplements and medicines.

In some embodiments, methods described herein further comprise the step of providing the report to the subject or to the subject's physician. In some embodiments, methods described herein further comprise providing a personalized nutritional composition to the subject based upon the report (e.g., the recommendations made in the report).

### Nutritional Compositions

Aspects of the disclosure relate to nutritional compositions. A “nutritional composition” generally refers to a composition comprising one or more vitamins, minerals, prebiotics, probiotics, natural extracts, amino acids, peptides, proteins, or any combination thereof, that does not require a doctor's prescription to obtain.

In some embodiments, the components of a nutritional composition are personalized (e.g., selected based upon computer-based modeling using the SNPs detected in one or more genes of a subject). In some embodiments, nutritional compositions are useful for dietary management of immune health of a subject. In some embodiments, ingestion of a nutritional composition by a subject reduces the risk that the subject will develop a pathogenic infection or reduces the risk that the subject will suffer complications because of a pathogenic infection. In some embodiments, nutritional compositions described herein are useful for reducing the risk of a subject of being infected with SARS-CoV-2 or a variant thereof. In some embodiments, nutritional compositions described herein are useful for reducing the risk of a subject of suffering from severe complications of SARS-CoV-2 (e.g., trouble breathing, persistent pain or pressure in the chest, lips turning blue, acute respiratory distress syndrome (ARDS), thromboembolic complications, acute cardiac, kidney, and liver injury, cardiac arrhythmias, rhabdomyolysis, coagulopathy, and shock).

The form of a nutritional composition may vary. In some embodiments, a nutritional composition is a solid. In some embodiments, a nutritional composition is a powder, pellet, bead, tablet capsule, microparticle, or nanoparticle. In some embodiments, a nutritional composition is a gel or a liquid. Examples of gels and liquids include but are not limited to solutions (e.g., aqueous solutions), suspensions, emulsions, elixirs, syrups, drops, sprays, draughts, and parenteral preparations. In some embodiments, a nutritional composition is provided as an intravenous (IV) drip. In some embodiments, a nutritional composition comprises one or more additional pharmaceutically acceptable excipients, for example as described by REMINGTON'S PHARMACEUTICAL SCIENCES, 18th Ed. (1990).

Aspects of the disclosure relate to nutritional compositions comprising selenium, vitamin D3, zinc, vitamin C, melatonin, and N-acetylcysteine (NAC). Without wishing to be bound by any particular theory, the combination of selenium, vitamin C, and NAC in a nutritional composition, in some embodiments, provides an increase in immune health of a subject, for example by increasing concentrations of AAT in the subject and/or increasing antioxidant concentrations in a subject (e.g., a subject in which one or more SNPs have been identified according to methods described herein).

The amount of selenium in a nutritional composition may vary. In some embodiments, the amount of selenium ranges from about 1 μg to about 500 μg. In some embodiments, the amount of selenium ranges from about 1 μg to about 5 μg, about 5 μg to about 20 μg, about 15 μg to about 50 μg, about 30 μg to about 100 μg, about 50 μg to about 120 μg, about 100 μg to about 150 μg, about 125 μg to about 175 μg, about 150 μg to about 200 μg, about 175 μg to about 250 μg, about 200 μg to about 300 μg, about 250 μg to about 375 μg, about 325 μg to about 450 μg, or about 400 μg to about 500 μg. In some embodiments, the amount of selenium in a nutritional composition is more than 500 μg. In some embodiments, the selenium comprises selenized yeast (also referred to as selenium yeast).

The amount of vitamin D3 in a nutritional composition may vary. In some embodiments, the amount of vitamin D3 ranges from about 1 μg to about 100 μg. In some embodiments, the amount of vitamin D3 ranges from about 1 μg to about 5 μg, about 5 μg to about 10 μg, about 7 μg to about 15 μg, about 10 μg to about 20 μg, about 15 μg to about 30 μg, about 20 μg to about 45 μg, about 30 μg to about 60 μg, about 50 μg to about 75 μg, about 60 μg to about 80 μg, or about 70 μg to about 100 μg. In some embodiments, the amount of vitamin D3 in a nutritional composition is more than 100 μg. In some embodiments, the amount of vitamin D3 ranges from about 1000 IU to about 5000 IU. In some embodiments, the amount of vitamin D3 ranges from about 1000 IU to about 2000 IU, about 1500 IU to about 2500 IU, about 3000 IU to about 4500 IU, or about 3500 IU to about 5000 IU. In some embodiments, the amount of vitamin D3 is more than 5000 IU.

The amount of N-acetylcysteine (NAC) in a nutritional composition may vary. In some embodiments, the amount of NAC ranges from about 1 mg to about 1200 mg. In some embodiments, the amount of NAC ranges from about 1 mg to about 10 mg, about 5 mg to about 20 mg, about 10 mg, to about 100 mg, about 50 mg to about 500 mg, about 400 mg to about 900 mg, or about 800 mg to about 1200 mg. In some embodiments, the amount of NAC in a nutritional composition is more than 1200 mg.

The amount of zinc in a nutritional composition may vary. In some embodiments, the amount of zinc ranges from about 1 μg to about 50 mg. In some embodiments, the amount of zinc ranges from about 1 mg to about 5 mg, about 5 mg to about 20 mg, or about 15 mg to about 50 mg. In some embodiments, the amount of zinc in a nutritional composition is more than 50 mg.

The amount of melatonin in a nutritional composition may vary. In some embodiments, the amount of melatonin ranges from about 1 mg to about 12 mg. In some embodiments, the amount of melatonin ranges from about 1 mg to about 10 mg, or about 5 mg to about 20 mg. In some embodiments, the amount of melatonin is about 1 mg, 2 mg, 3 mg, 4 mg, 5 mg, 6 mg, 7 mg, 8 mg, 9 mg, 10 mg, 11 mg, or 12 mg. In some embodiments, the amount of melatonin in a nutritional composition is more than 12 mg.

The amount of vitamin C in a nutritional composition may vary. In some embodiments, the amount of vitamin C ranges from about 1 μg to about 1200 μg. In some embodiments, the amount of vitamin C ranges from about 1 μg to about 5 μg, about 5 μg to about 20 μg, about 15 μg to about 50 μg, about 30 μg to about 100 μg, about 50 μg to about 120 μg, about 100 μg to about 150 μg, about 125 μg to about 175 μg, about 150 μg to about 200 μg, about 175 μg to about 250 μg, about 200 μg to about 300 μg, about 250 μg to about 375 μg, about 325 μg to about 450 μg, about 400 μg to about 500 μg, about 350 μg to about 700 μg, or about 500 μg to about 1200 μg. In some embodiments, the amount of vitamin C in a nutritional composition is more than 1200 μg (e.g., 3 mg, 4 mg, 5 mg, 6 mg, 7 mg, 8 mg, or more).

In some embodiments, a nutritional composition comprises quercetin. The amount of quercetin may vary. In some embodiments, the amount of quercetin ranges from about 1 mg to about 1000 mg. In some embodiments, the amount of quercetin ranges from about 1 mg to about 10 mg, about 5 mg to about 20 mg, about 10 mg, to about 100 mg, about 50 mg to about 500 mg, about 400 mg to about 900 mg, or about 800 mg to about 1000 mg. In some embodiments, the amount of quercetin in a nutritional composition is more than 1000 mg.

In some embodiments, a nutritional composition comprises lysine. In some embodiments, the lysine comprises L-Lysine. The amount of lysine (e.g., L-lysine) in a nutritional composition may vary. In some embodiments, the amount of lysine in a nutritional composition ranges from about 1 mg to about 1000 mg. In some embodiments, the amount of lysine ranges from about 1 mg to about 10 mg, about 5 mg to about 20 mg, about 10 mg, to about 100 mg, about 50 mg to about 500 mg, about 400 mg to about 900 mg, or about 800 mg to about 1000 mg. In some embodiments, the amount of lysine in a nutritional composition is more than 1000 mg.

In some embodiments, a nutritional composition comprises Coenzyme Q10. The amount of Coenzyme Q10 in a nutritional composition may vary. In some embodiments, the amount of Coenzyme Q10 ranges from about 1 mg to about 20 mg. In some embodiments, the amount of Coenzyme Q10 ranges from about 1 mg to about 5 mg, about 3 mg to about 7 mg, about 5 mg to about 10 mg, about 8 mg to about 14 mg, about 10 mg to about 15 mg, or about 12 mg to about 20 mg. In some embodiments, the amount of Coenzyme Q10 in a nutritional composition is more than 20 mg.

In some embodiments, a nutritional composition comprises L-glutathione. The amount of L-glutathione in a nutritional composition may vary. In some embodiments, the amount of L-glutathione in a nutritional composition ranges from about 1 mg to about 100 mg. In some embodiments, the amount of L-glutathione ranges from about 1 mg to about 10 mg, about 5 mg to about 20 mg, about 10 mg, to about 100 mg, about 50 mg to about 500 mg, about 400 mg to about 900 mg, or about 800 mg to about 1000 mg. In some embodiments, the amount of L-glutathione in a nutritional composition is more than 1000 mg.

In some embodiments, a nutritional composition comprises pantothenic acid (Vitamin B5). The amount of pantothenic acid in a nutritional composition may vary. In some embodiments, the amount of pantothenic acid ranges from about 1 mg to about 20 mg. In some embodiments, the amount of pantothenic acid ranges from about 1 mg to about 5 mg, about 2 mg to about 8 mg, about 5 mg to about 12 mg, about 8 mg to about 15 mg, or about 12 mg to about 20 mg. In some embodiments, the amount of pantothenic acid in a nutritional composition is more than 20 mg.

In some embodiments, a nutritional composition comprises one or more (e.g., 1, 2, 3, 4, 5, or more) probiotic agents. Examples of probiotic agents include but are not limited to certain bacteria species (e.g., Lactobacillus, Lactococcus, Leuconostoc, Pediococcus, Bifidobacterium, Bacillus, etc.) and certain yeast (e.g., Saccharomyces). In some embodiments, a nutritional composition comprises one or more of the following probiotic agents: Lactobacillus rhamnosus, Bifidobacterium bifidum, Bifidobacterium lactic, or any combination thereof. The amount of each probiotic agent in a nutritional composition may vary. In some embodiments, the amount of each probiotic agent ranges from about 1.0 billion colony forming units (CFU) to about 10.0 billion CFU. In some embodiments, the amount of each probiotic agent ranges from about 2.0 billion CFU to about 8.0 billion CFU. In some embodiments, the amount of each probiotic agent ranges from about 4.0 billion CFU to about 6.0 billion CFU.

In some embodiments, a nutritional composition comprises one or more (e.g., 1, 2, 3, 4, 5, or more) natural plant extracts. Examples of natural plant extracts include Echinacea purpurea extract, Elderberry fruit extract, garlic (Allium sativum) extract, green tea (Camellia sinensis) extract, ginger (Zingiber officinale) extract, turmeric (Curcuma longa) extract, evening primrose (Oenothera biennis) extract, flax seed (Linum usitatissimum) extract, tea tree (Melaleuca alternifolia) extract, grapeseed (Vitis vimfera) extract, etc. The amount of each natural plant extract in a composition may vary from about 100 mg to about 500 mg. In some embodiments, the amount of each plant extract ranges from about 100 mg to about 200 mg, 150 mg to about 300 mg, about 200 mg to about 400 mg, or about 350 mg to about 500 mg. In some embodiments, the amount of each plant extract is more than 500 mg.

In some embodiments, the nutritional composition comprises (or consists of) the following formula: 200 μg selenium yeast, 25 μg vitamin D (1000 IU vitamin D), 50 mg zinc, 1 mg vitamin C, 3 mg melatonin, 400 mg NAC, 200 mg Elderberry fruit extract, 200 mg Echinacea purpurea plant extract, 5.0 billion CFU Bifidobacterium bifidum, 5.0 billion CFU Bifidobacterium lactic, and 6.0 billion CFU Lactobacillus rhamnosus.

Aspects of the disclosure are related to nutritional compositions that are useful for management of Long Haul COVID. In some embodiments, a subject having “Long Haul COVID” refers to a subject having one or more signs or symptoms of SARS-CoV-2 infection lasting more than one month after infection with a SARS-CoV-2 virus. Examples of signs and symptoms of Long Haul COVID include but are not limited to difficulty breathing or shortness of breath, tiredness or fatigue, symptoms that get worse after physical or mental activities (also known as post-exertional malaise), difficulty thinking or concentrating (sometimes referred to as “brain fog”), cough, chest or stomach pain, headache, fast-beating or pounding heart (also known as heart palpitations), joint or muscle pain, pins-and-needles feeling, diarrhea, sleep problems, fever, dizziness on standing (lightheadedness), rash, mood changes, change in smell or taste, or changes in menstrual period cycles. In some embodiments, a subject having Long Haul COVID exhibits one or more signs or symptoms for greater than one month, two months, six months, one year, or more than one year after being infected with a SARS-CoV-2 virus.

In some embodiments, the disclosure provides liquid (e.g., aqueous) nutritional compositions that are useful for management of Long Haul COVID. In some embodiments, the liquid nutritional compositions are formulated for intravenous (IV) delivery to a subject. In some embodiments, the liquid nutritional compositions comprise one or more of the following ingredients: water, ascorbic acid, B-complex, Calcium gluconate, Dexpanthenol, Folic acid, Magnesium chloride, Methylcobalamin, Multi-trace 4, Potassium chloride, Selenium, Sodium chloride (also solution, 0.9%), Zinc sulfate, NAC, L-carnitine, Glutathione, and Phosphatidylcholine. In some embodiments, the components of a liquid nutritional composition are delivered to a subject in more than one (e.g., 1, 2, 3, or more than 3) intravenous injection bags. In some embodiments, the first IV bag comprises water, ascorbic acid, B-complex, Calcium gluconate, Dexpanthenol, Folic acid, Magnesium chloride, Methylcobalamin, Multi-trace 4, Potassium chloride, Selenium, Sodium chloride (also solution, 0.9%), Zinc sulfate, NAC, and L-carnitine. In some embodiments, the second IV bag comprises a sodium chloride aqueous solution (e.g., 0.9% saline solution) and Glutathione. In some embodiments, the third IV bag comprises liquid phosphatidylcholine.

The amount of vitamin C (e.g., ascorbic acid) in a liquid composition may vary. In some embodiments, the amount of ascorbic acid (e.g., vitamin C) ranges from about 100 mg to about 8000 mg. In some embodiments, the amount of ascorbic acid ranges from about 100 mg to about 500 mg, about 200 mg to about 800 mg, about 400 mg to about 1200 mg, about 1000 mg to about 3000 mg, about 2000 mg to about 5000 mg, about 3000 mg to about 6000 mg, or about 4000 mg to about 8000 mg. In some embodiments, a liquid nutritional composition comprises more than 8000 mg ascorbic acid (e.g., vitamin C).

In some embodiments, B-complex comprises a mixture comprising one or more of the following B-vitamins: B-1 (thiamine), B-2 (riboflavin), B-3 (niacin), B-5 (pantothenic acid), B-6 (pyridoxine), B-7 (biotin), B-9 (folic acid), and B-12 (cobalamin). In some embodiments, B-complex comprises, per 1 ml: Thiamine Hydrochloride (B1) 100 mg, Riboflavin (B2) 5 mg (e.g., as Riboflavin 5′-Phosphate Sodium), Niacinamide, 100 mg, Pyridoxine Hydrochloride (B6) 10 mg, d-Panthenol 10 mg, and cyanocobalamin (B12) 100 μg. The amount of B-complex in a liquid composition may vary. In some embodiments, the amount of B-complex ranges from about 1 ml to about 5 ml.

The amount of calcium gluconate in a liquid composition may vary. In some embodiments, the amount of calcium gluconate ranges from about 10 mg to about 500 mg. In some embodiments, the amount of calcium gluconate ranges from about 10 mg to about 100 mg, about 50 mg to about 250 mg, about 100 mg to about 300 mg, about 200 mg to 400 mg, or about 300 mg to about 500 mg.

The amount of Dexpanthenol in a liquid composition may vary. In some embodiments, the amount of Dexpanthenol ranges from about 10 mg to about 500 mg. In some embodiments, the amount of Dexpanthenol ranges from about 10 mg to about 100 mg, about 50 mg to about 250 mg, about 100 mg to about 300 mg, about 200 mg to 400 mg, or about 300 mg to about 500 mg.

The amount of folic acid in a liquid composition may vary. In some embodiments, the amount of folic acid in a liquid composition is about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg.

The amount of magnesium chloride in a liquid composition may vary. In some embodiments, the amount of magnesium chloride ranges from about 10 mg to about 500 mg. In some embodiments, the amount of magnesium chloride ranges from about 10 mg to about 100 mg, about 50 mg to about 250 mg, about 100 mg to about 300 mg, about 200 mg to 400 mg, or about 300 mg to about 500 mg.

The amount of methylcobalamin in a liquid composition may vary. In some embodiments, the amount of methylcobalamin ranges from about 1 μg to about 5 μg, about 5 μg to about 20 μg, about 15 μg to about 50 μg, about 30 μg to about 100 μg, about 50 μg to about 120 μg, about 100 μg to about 150 μg, about 125 μg to about 175 μg, about 150 μg to about 200 μg, about 175 μg to about 250 μg, about 200 μg to about 300 μg, about 250 μg to about 375 μg, about 325 μg to about 450 μg, about 400 μg to about 500 μg, about 350 μg to about 700 μg, or about 500 μg to about 1200 μg.

As used herein, “Multi-trace 4” refers to a composition comprising zinc (5 mg/ml), copper (1 mg/ml), manganese (0.5 mg/ml) and Chromium (10 μg/ml). The amount of Multi-trace 4 in a liquid composition may vary. In some embodiments, the amount of Multi-trace 4 ranges from about 1 ml to about 5 ml. In some embodiments, a liquid composition comprises 1 ml, 2 ml, 3 ml, 4 ml, or 5 ml Multi-trace 4.

The amount of potassium chloride in a liquid composition may vary. In some embodiments, the amount of potassium chloride in a liquid composition ranges from about 0.5mEq k+/ml to about 3mEq k+/ml.

The amount of selenium in a liquid composition may vary. In some embodiments, the amount of selenium ranges from about 1 μg to about 500 μg. In some embodiments, the amount of selenium ranges from about 1 μg to about 5 μg, about 5 μg to about 20 μg, about 15 μg to about 50 μg, about 30 μg to about 100 μg, about 50 μg to about 120 μg, about 100 μg to about 150 μg, about 125 μg to about 175 μg, about 150 μg to about 200 μg, about 175 μg to about 250 μg, about 200 μg to about 300 μg, about 250 μg to about 375 μg, about 325 μg to about 450 μg, or about 400 μg to about 500 μg. In some embodiments, the amount of selenium in a nutritional composition is more than 500 μg.

The amount of sodium chloride (e.g., saline) in a liquid composition may vary. In some embodiments, the sodium chloride is provided as a saline solution, e.g., a 0.9% saline solution. In some embodiments, the sodium chloride is provided as an 8.4% solution. In some embodiments, the saline solution is present in an amount ranging from about 1 ml to about 500 ml. In some embodiments, the saline solution is sterile.

The amount of zinc sulfate in a liquid composition may vary. In some embodiments, the amount of zinc sulfate ranges from about 1 μg to about 50 mg. In some embodiments, the amount of zinc sulfate ranges from about 1 mg to about 5 mg, about 5 mg to about 20 mg, or about 15 mg to about 50 mg. In some embodiments, the amount of zinc sulfate in a liquid nutritional composition is more than 50 mg.

The amount of NAC in a liquid composition may vary. In some embodiments, the amount of NAC ranges from about 1 mg to about 1200 mg. In some embodiments, the amount of NAC ranges from about 1 mg to about 10 mg, about 5 mg to about 20 mg, about 10 mg, to about 100 mg, about 50 mg to about 500 mg, about 400 mg to about 900 mg, or about 800 mg to about 1200 mg. In some embodiments, the amount of NAC in a nutritional composition is more than 1200 mg.

The amount of L-carnitine in a liquid composition may vary. In some embodiments, the amount of L-carnitine ranges from about 1 mg to about 1200 mg. In some embodiments, the amount of L-carnitine ranges from about 1 mg to about 10 mg, about 5 mg to about 20 mg, about 10 mg, to about 100 mg, about 50 mg to about 500 mg, about 400 mg to about 900 mg, or about 800 mg to about 1200 mg. In some embodiments, the amount of L-carnitine in a nutritional composition is more than 1200 mg.

The amount of glutathione in a liquid composition may vary. In some embodiments, the amount of glutathione ranges from about 100 mg to about 8000 mg. In some embodiments, the amount of glutathione from about 100 mg to about 500 mg, about 200 mg to about 800 mg, about 400 mg to about 1200 mg, about 1000 mg to about 3000 mg, about 2000 mg to about 5000 mg, about 3000 mg to about 6000 mg, or about 4000 mg to about 8000 mg. In some embodiments, a liquid nutritional composition comprises more than 8000 mg glutathione.

The amount of phosphatidylcholine in a liquid composition may vary. In some embodiments, the amount of phosphatidylcholine ranges from about 10 mg to about 500 mg. In some embodiments, the amount of phosphatidylcholine ranges from about 10 mg to about 100 mg, about 50 mg to about 250 mg, about 100 mg to about 300 mg, about 200 mg to 400 mg, or about 300 mg to about 500 mg.

### EXAMPLES

### Example 1

This example describes a combination of genetic testing with AI-driven probabilistic models in order to provide nutritional and dietary recommendations to a subject. In some embodiments, the nutritional recommendations provided to the subject aid in dietary management of immune health in the subject.

Briefly, a biological sample, such as a blood sample or cheek swab, is obtained from a subject. Nucleic acids (e.g., DNA, RNA, etc.) are extracted from the sample and a sequencing reaction is performed. The sequencing data is then analyzed to identify the presence of single nucleotide polymorphisms (SNPs) in certain genes of the subject, for example those listed in Table 1. The data obtained from the SNP identification is then modeled using an artificial intelligence (AI) probabilistic model in order to provide an individualized summary of supplement, dietary, lifestyle, and follow-up interventions supported by the genetic results. The report contains recommended nutritional supplements that target the underlying biological mechanisms associated with the SNPs.

TABLE 1

AAT
ABC7A
ACE
AKT1
ANK3
APOE
BDNF
BNP
CACNA1C
CD33
CHRNA5/A3
CLOCK
COMT
CRHR1
CRP
FKBP5
FTP
FUT2
HDAC9
HLA-DQ2.2/3/2.5/8
HLA-DQB1
IL6
LRP1
LRRK2
MC4R
MEIS1
MIR-181
MTHFR
OXTR
PCSK9
PPARG
SMARCA4
TREM2

By way of an example, if the subject has one or more SNPs in AAT the subject is identified as potentially having A1AT deficiency and thus at an increased risk of developing a pathogenic infection, such as SARS-CoV-2 infection. Certain nutritional supplements, such as selenium, have been observed to change pre-serum amyloid P factors, as well as alpha-1-antitrypsin (A1AT) serum concentration in healthy subjects. Thus, the report for this subject may include a recommendation to ingest a selenium supplement, such as selenized yeast. The report may also recommend additional nutritional supplements, such as vitamin D3, zinc, vitamin C, melatonin, and N-acetylcysteine (NAC) for that individual. The report may also recommend additional dietary aids, such as one or more probiotics.

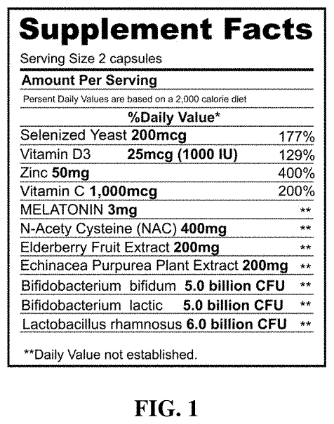

The personalized nutritional supplement recommended by the report may be produced. In some embodiments, the supplement is a solid (e.g., in a tablet or capsule form), and comprises the ingredients listed in the label shown in FIG. 1.

### Example 2

Single nucleotide polymorphisms, frequently called SNPs, which are the most common type of genetic variation among people. SNPs occur normally throughout a person's DNA, for example almost once in every 1,000 nucleotides on average, which means there are roughly 4 to 5 million SNPs in a person's genome. These variations may be unique or occur in many individuals; more than 100 million SNPs have been identified in populations around the world. Most commonly, these variations are found in the DNA between genes. They can act as biological markers, helping scientists locate genes that are associated with disease. When SNPs occur within a gene or in a regulatory region near a gene, they may play a more direct role in disease by affecting the gene's function.

Most SNPs have no effect on health or development. Some of these genetic differences, however, have proven to be particularly important in the study of human health. Researchers have found SNPs that may help predict an individual's response to certain drugs, susceptibility to environmental factors such as toxins, and risk of developing particular diseases. SNPs can also be used to track the inheritance of disease genes within families.

This example describes detection of SNPs for the purpose of providing patients and their subjects with relevant genetic results for guiding lifestyle, diet, and supplement choices with the goal of improving overall health (e.g., by means of dietary management of immune health). Briefly, SNPs in 32 genes (e.g., genes listed in Table 1) are classified into at least six ‘domains’ and are identified. A report containing a summary of evidence-based dietary recommendations is provided based upon the identified SNPs of each domain. In some embodiments, genetic results (e.g., identified SNPs) that do not have any known health impact are listed at the end of the report. Examples of the six ‘domains’ are described below.

### 1. Cardiometabolic

Several genetic risk factors have been identified, which disrupt biological pathways involved with lipid metabolism, hemostasis, and inflammatory processes leading to an overall increased risk of diabetes, heart disease, mood disorders, and cognitive decline. Preventative care modalities have also been identified that modulate the risks associated with these genetic polymorphisms. Examples of genes and SNPs associated with this domain include but are not limited to ApoE, BNP, FTO, MC4R, PCSK9, PPARG, SMARCA4, HDAC9, and ACE.

### 2. Stress and Emotional Wellbeing

The ‘fight or flight’ response to stress involves complex hormonal signaling loops that modulate an appropriate response to environmental threats. Genetic risk factors have been identified in recent years that impact the regulation process, leading to an increased risk of physical and psychological morbidity. Concurrently, multiple treatment modalities have been identified to target these underlying mechanisms of dysregulated stress response. Examples of genes and SNPs associated with this domain include but are not limited to iRPI, AKTI, CLOCK, CACNA1C, OXTR, COMT, CRHR1, MTHFR. BDNF, CHRNA5/3, and ANK3.

### 3. Cognition and Mental Acuity

Memory, focus, perception, and mood are manifested through intricate, interconnected systems. Disruptions of these systems, due to a combination of environmental factors and genetic predisposition, has been shown to significantly increase the risks of cognitive clouding and decline. Determining the root causal mechanisms of these disruptions allow a targeted approach to improve executive function, working memory and the slowing of the relentless march of cognitive decline. Examples of genes and SNPs associated with this domain include but are not limited to ApoE, CD33, ABCA7, LRRK2, and TREM2.

### 4. Inflammation

Inflammation is a finely tuned, dynamic process required for immune surveillance, tissue repair, and regeneration after injury. However, dysregulation of these processes due to environmental and genetic risk factors underlies many complex diseases including infection, obesity, neurodegenerative disease, atherosclerosis, and cancer. Examples of genes and SNPs associated with this domain include but are not limited to CRP, and IL6.

### 5. GI and Immune

There is an intricate network formed between the underlying biology of the gastrointestinal tract and the intracellular signaling of the immune system. This network involves the gut microbiota which impact nutrient absorption and immune-mediated metabolic function. Genetic variants, which ultimately lead to dysbiosis, or perturbation in intestinal microbiota, have been associated with the development and progression of inflammatory disorders. Examples of genes and SNPs associated with this domain include but are not limited to FPLADQ2. 2/4/2.5/8, CRP, and FVT2.

### 6. Sleep

Sleep is an essential feature of brain plasticity—the brain's ability to evolve and adapt. Normal sleep patterns promote hormonal balance, tissue regeneration, mood stability, and executive function. Sleep dysfunction leads to autonomic dysregulation changes in cortisol metabolism, and immune dysfunction. Identification of underlying mechanisms disrupting circadian rhythms or excitatory signaling is essential to selecting appropriate interventions to increase overall health and wellbeing. Examples of genes and SNPs associated with this domain include but are not limited to CLOCK, CACNA1C, HLADQBI, and MEIS1.

SNPs may be detected by any suitable method, for example as described below. DNA is extracted from a biological sample (e.g., saliva, blood, buccal swab, etc.). A TaqMan-based genotyping assay may be used to detect (e.g., genotype) alleles of certain genes (detect Z and S alleles of AAT gene). Alternatively, PCR-based sequencing of an entire coding region, intron/exon boundaries, and known pathogenic variants (e.g., as described in the HGMD version 2018.1; www.hgmd.cf.ac.uk/ac/index.php) in the promoter and deep intronic regions of the specified gene are detected. The genotyping tests and/or DNA sequencing tests are typically performed by a CLIA qualified laboratory. In some embodiments, the genotyping and/or DNA sequencing tests are classified as Lab-Developed Tests (LDTs).

In some embodiments, one or more of the following SNPs are detected: ABCA7 (rs3764650), ACE (rs4343), AKT1 (rs2494732), ANK3 (rs10994336), APOE (rs429358, rs4712), BDNF (rs6265), BNP (rs198389), CACNA1C (rs1006737), CD33 (rs3865444), CHRNA5/3 (rs16969968, rs578776, rs588765), CLOCK (rs1801260), COMT (rs4680), CRHR1 (rs110402), CRP (rs1130864), FKBP5 (rs1360780), FTO (rs9939609), FUT2 (rs1047781, rs601338), HDAC9 (rs11984041), HLA-DQB1*06.02; HLA-DQ2.5 (rs2187668), HLA-DQ2.2 (rs2395182, rs7775228), HLADQ4 (rs4713586), HLA DQ8 (rs7454108), IL6 (rs1800795), LRRK2 (rs34637584), LRP1 (rs11172113), MC4R (rs17782313), MEIS1 (rs2300478), MIR-181 (rs322931), MTHFR (rs1801131, rs1801133), OXTR (rs53576), PCSK9 (rs11206510), PPARG (rs1801282), SMARCA4 (LDLR; rs1122608), and TREM2 (rs75932628).

### Example 3

This example describes a supplement formulation compounded for intravenous (IV) delivery that includes vitamins and supplements to provide support and relief to COVID-19 Long Haulers having breathing, neurological and other symptoms. In some embodiments, the formulation is administered weekly, for example during appointments in a physician's office or a medical facility.

The IV formulation comprises three IV bags having the components shown in Table 2.

TABLE 2

| | Component | Amount | | Volume | |
|---|---|---|---|---|---|
| 1st Bag | | | | | |
| 1 | Sterile water | | | 250 | ml |
| 2 | Ascorbic acid | 500 | mg/ml | 15 | ml |
| 3 | B-Complex | | | 1 | ml |
| 4 | Calcium Gluconate | 100 | mg/ml | 3 | ml |
| 5 | Dexpanthenol | 250 | mg/ml | 1 | ml |
| 6 | Folic acid | 5 | mg/ml | 1 | ml |
| 7 | Magnesium Chloride | 200 | mg/ml | 2 | ml |
| 8 | Methylcobalamin | 1000 | mcg/ml | 1 | ml |
| 9 | Multi trace-4 | | | 1 | ml |
| 10 | Potassium chloride | 2 | mEq k+/ml | 1 | ml |
| 11 | Selenium | 40 | mcg/ml | 1 | ml |
| 12 | Sodium chloride | 8.40% | | 2 | ml |
| 13 | Zinc sulfate | 10 | mg/ml | 1 | ml |
| 14 | NAC | 100 | mg/ml | 1 | ml |
| 15 | L-Carnitine | 100 | mg/ml | 1 | ml |
| 2nd bag | Sodium chloride solution | 0.90% | | 100 | ml |
| | Glutathione | 200 | mg/ml | 10 | ml |
| 3rd | Phosphatidylcholine | 250 | mg | 1 | Ampule |

### Example 4

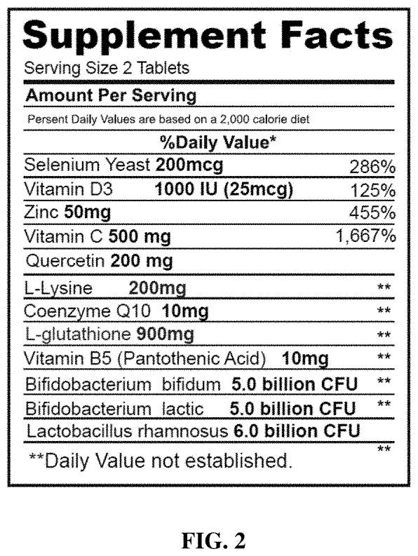

This example describes an oral nutritional supplement for use in dietary management of COVID-19 infection in a subject. The nutritional supplement is in a solid form, optionally formulated as a tablet. In some embodiments, the nutritional supplement comprises the components listed in the label shown in FIG. 2.

What is claimed is:

1. A method for dietary management of immune health in a subject, the method comprising:
(i) detecting in nucleic acid sequencing data obtained from the subject one or more single nucleotide polymorphisms (SNPs) in an alpha-1 antitrypsin (A1AT) gene; and
(ii) providing a report indicating that the subject should ingest a selenium supplement as part of the subject's daily diet.

2. A method for diagnosing a subject as having an increased risk of developing a pathogenic infection the method comprising:
(i) detecting in nucleic acid sequencing data obtained from the subject one or more single nucleotide polymorphisms (SNPs) in:
(a) an alpha-1 antitrypsin (A1AT) gene; and
(b) two or more genes selected from ABCA7, ACE, AKT1, ANK3, APOE, BDNF, BNP, CACNA1C, CD33, CHRNA5/A3, CLOCK, COMT, CRHR1, CRP, FKBP5, FTP, FUT2, HDAC9, HLA-DQ2.2/3/2.5/8, HLA-DQB1, IL6, LRP1, LRRK2, MC4R, MEIS1, MIR-181, MTHFR, OXTR, PCSK9, PPARG, SMARCA4, and TREM2; and
(ii) identifying the subject as having an increased risk of developing a pathogenic infection when the SNP in AAT is a SNP associated with AAT deficiency and when the SNP.

3. The method of claim 1 or 2, wherein the subject is a human.

4. The method of any one of claims 1 to 3, wherein the SNP in the A1AT gene results in the subject expressing a PI*S or PI*Z A1AT protein, optionally wherein the SNPs in two or more genes are selected from: ABCA7 (rs3764650), ACE (rs4343), AKT1 (rs2494732), ANK3 (rs10994336), APOE (rs429358, rs4712), BDNF (rs6265), BNP (rs198389), CACNA1C (rs1006737), CD33 (rs3865444), CHRNA5/3 (rs16969968, rs578776, rs588765), CLOCK (rs1801260), COMT (rs4680), CRHR1 (rs110402), CRP (rs1130864), FKBP5 (rs1360780), FTO (rs9939609), FUT2 (rs1047781, rs601338), HDAC9 (rs11984041), HLA-DQB1*06.02; HLA-DQ2.5 (rs2187668), HLA-DQ2.2 (rs2395182, rs7775228), HLADQ4 (rs4713586), HLA DQ8 (rs7454108), IL6 (rs1800795), LRRK2 (rs34637584), LRP1 (rs11172113), MC4R (rs17782313), MEIS1 (rs2300478), MIR-181 (rs322931), MTHFR (rs1801131, rs1801133), OXTR (rs53576), PCSK9 (rs11206510), PPARG (rs1801282), SMARCA4 LDLR; rs1122608), and TREM2 (rs75932628).

5. The method of any one of claims 1 to 4, wherein the nucleic acid sequencing data comprises DNA expression data, RNA expression data, whole exome sequencing expression data, or microarray data.

6. The method of any one of claims 1 to 5, wherein the nucleic acid sequencing data is obtained from a biological sample obtained from the subject.

7. The method of claim 6, wherein the biological sample comprises blood, saliva, urine, feces, semen, or a tissue sample.

8. The method of any one of claims 1 to 7, wherein SNPs are detected in 3, 4, 5, 6, 7, 8, 9, 10, 11, 12, 13, 14, 15, 16, 17, 18, 19, 20, 21, 22, 23, 24, 25, 26, 27, 28, 29, or 30 of the genes listed in step (i)(b).

9. The method of any one of claims 1 to 8, wherein the method further comprises producing a report indicating that the subject should be administered a nutritional supplement comprising selenium based upon the detecting of the A1AT SNP in step (ii).

10. The method of any one of claims 1 to 9, wherein the selenium comprises selenized yeast (selenium yeast).

11. The method of claim 9 or 10, wherein the nutritional supplement further comprises one or more of the following: vitamin D, N-acetylcysteine, zinc, melatonin, and vitamin C.

12. The method of any one of claims 1 to 11, further comprising providing to the subject a nutritional supplement comprising selenium.

13. The method of any one of claims 1 to 12, further comprising administering a nutritional supplement comprising selenium to the subject.

14. A nutritional composition comprising selenium, vitamin D3, zinc, vitamin C, melatonin, and N-acetylcysteine (NAC).

15. The nutritional composition of claim 14, wherein the amount of selenium ranges from about 1 μg to about 500 μg.

16. The nutritional composition of claim 14 or 15, where the selenium comprises selenized yeast (selenium yeast).

17. The nutritional composition of any one of claims 14 to 16, wherein the amount of vitamin D3 ranges from about 1 μg to about 100 μg, or about 1000 IU to about 5000 IU.

18. The nutritional composition of any one of claims 14 to 17, wherein the amount of NAC ranges from about 1 mg to about 1200 mg

19. The nutritional composition of any one of claims 14 to 18, wherein the amount of zinc ranges from about 1 mg to about 100 mg.

20. The nutritional composition of any one of claims 14 to 19, wherein the amount of melatonin ranges from about 1 mg to about 12 mg.

21. The nutritional composition of any one of claims 14 to 20, wherein the amount of vitamin C ranges from about 1 μg to about 1200 μg.

22. The nutritional composition of any one of claims 14 to 21, wherein the composition is a solid, optionally wherein the composition is a powder.

23. The nutritional composition of any one of claims 14 to 22, further comprising one or more pharmaceutically acceptable excipients.

24. The nutritional composition of any one of claims 14 to 23, comprising the formula listed in FIG. 1.

25. A method of dietary management of a respiratory disease (e.g., SARS-CoV-2 or COPD), the method comprising administering the nutritional composition of any one of claims 14 to 24 to a subject in need thereof.

26. The method of claim 25, wherein the subject has or is suspected of having an infection with SARS-CoV-2 or COPD.

27. The method of claim 25 or 26, wherein the subject is known to have been exposed to SARS-CoV-2.

28. The method of claim 25, wherein the subject is at risk of being exposed to SARS-CoV-2.

29. The method of any one of claims 25 to 28, wherein the subject has one or more mutations in an A1AT gene.

30. The method of any one of claims 25 to 28, wherein the subject has A1AT deficiency.

31. A nutritional composition comprising selenium, vitamin D3, zinc, vitamin C, quercetin, Coenzyme Q10, L-lysine, L-glutathione, and vitamin B5.

32. The nutritional composition of claim 31, further comprising one or more probiotic bacteria.

33. The nutritional composition of claim 31 or 32, wherein the composition is a solid, optionally wherein the composition is a powder or a tablet.

34. The nutritional composition of any one of claims 31 to 33, comprising the formula listed in FIG. 2.

35. A method for dietary management of a respiratory disease (e.g., SARS-CoV-2 or COPD), the method comprising administering the nutritional composition of any one of claims 31 to 34 to a subject in need thereof.

36. A liquid nutritional composition comprising water, ascorbic acid, B-complex, Calcium gluconate, Dexpanthenol, Folic acid, Magnesium chloride, Methylcobalamin, Multi-trace 4, Potassium chloride, Selenium, Sodium chloride (also solution, 0.9%), Zinc sulfate, NAC, and L-carnitine.

37. The liquid nutritional composition of claim 36, further comprising glutathione.

38. The liquid nutritional composition of claim 37 further comprising phosphatidylcholine.

39. The liquid nutritional composition of any one of claims 36 to 38, wherein the composition is formulated for intravenous (IV) delivery.

40. The liquid nutritional composition of any one of claims 36 to 39 comprising the formula listed in Table 2.

41. A system for dietary management of a respiratory disease (e.g., SARS-CoV-2 or COPD) comprising:
(i) a first intravenous (IV) injection solution comprising B-complex, Calcium gluconate, Dexpanthenol, Folic acid, Magnesium chloride, Methylcobalamin, Multi-trace 4, Potassium chloride, Selenium, Sodium chloride (also solution, 0.9%), Zinc sulfate, NAC, and L-carnitine;
(ii) a second intravenous (IV) injection solution comprising glutathione; and
(iii) a third intravenous (IV) injection solution comprising phosphatidylcholine.

42. A method for dietary management of Long Haul COVID, the method comprising administering the nutritional composition of any one of claims 36 to 40, or the solutions of system of claim 41, to a subject in need thereof.
